(19) United States
(12) Patent Application Publication
(10) Pub. No.: US 2021/0060214 A1
(43) Pub. Date: Mar. 4, 2021

(54) ELECTROSPUN ANTI-ADHESION BARRIER

(71) Applicant: THE SECANT GROUP, LLC, Telford, PA (US)

(72) Inventors: Jeremy J. HARRIS, Doylestown, PA (US); Jared ELY, Quakertown, PA (US); Mevlut TASCAN, Breinigsville, PA (US); Peter D. GABRIELE, Frisco, TX (US); Todd CRUMBLING, Perkasie, PA (US)

(21) Appl. No.: 17/006,129

(22) Filed: Aug. 28, 2020

Related U.S. Application Data
(60) Provisional application No. 62/892,587, filed on Aug. 28, 2019.

(51) Int. Cl. A61L 31/04; A61L 31/14; D04H 1/728

(52) U.S. Cl. CPC A61L 31/041; A61L 31/14; D10B 2331/04; D10B 2321/06; D04H 1/728

(57) ABSTRACT

An article includes a fibrous mat of poly(glycerol sebacate) (PGS) resin and a resin of a hydrogel forming polymer, such as a polyvinyl alcohol (PVOH). Methods of making such articles include electrospinning a combination of PGS resin and PVOH resin to form nanofibers and depositing the nanofibers onto a surface to form the fibrous mat. The mat is suitable for a variety of medical uses, including as a barrier that can be deployed in surgical procedures.

### RELATED APPLICATIONS

This application claims priority to and the benefit of U.S. Application No. 62/892,587 filed Aug. 28, 2019 and which is hereby incorporated by referenced in its entirety.

### FIELD

This application is directed to electrospun materials and processes of forming such materials.

### BACKGROUND

Tissue adhesions are a major source of post-surgical complications and pain following abdominal, pelvic and cardiac procedures. Resorptive anti-adhesion barriers (or simply adhesion barriers, for short) are often placed as a part of surgery for patients undergoing abdominal, pelvic or cardiac procedures (both open and laparoscopic approaches) as an adjunct intended to reduce the incidence, extent and severity of postoperative adhesions between the abdominal wall and the under-lying viscera such as omentum, small bowel, bladder, and stomach, and between the uterus and surrounding structures such as fallopian tubes and ovaries, large bowel, and bladder and between the chest wall and the pericardium and/or cardiac tissue.

Other applications for adhesion barriers include gynecologic pelvic surgery, for example, by dry application to traumatized surfaces after meticulous hemostasis consistent with microsurgical principles to physically separate opposing tissue surfaces during the period of reperitonealization. Further applications include use in cardiac surgical procedures to reduce the incidence of adhesion formation between cardiac tissue and the sternum. Additionally, for cardiac procedures, there is often a need to preserve a plane of dissection for ease of access in the event of future procedures.

Conventional adhesion barriers are unsatisfactory for a variety of reasons. These include difficulty of deployment in laparoscopic procedures and poor handling after hydration, meaning they cannot be easily repositioned once wet. Additionally, some conventional adhesion barriers are contraindicated for bloody sites and/or sites prone to infection, reducing their ability to be used in certain surgical procedures.

What is needed is a barrier that prevents adhesion between adjacent tissues that overcomes these and other problems in the art.

### SUMMARY

Exemplary embodiments are directed to an article comprising a fibrous mat of poly(glycerol sebacate) (PGS) resin and a resin of a hydrogel forming polymer, such as polyvinyl alcohol (PVOH). Exemplary embodiments are also directed to methods of making such articles, including electrospinning a combination of PGS resin and PVOH resin to form nanofibers and depositing the nanofibers onto a surface to form the fibrous mat.

The mat, which may also be referred to herein as a film, has a variety of uses and in some embodiments provides a barrier that can be deployed in both open and laparoscopic procedures, is capable of use in wet and/or bloody sites in addition to dry sites, and provides antimicrobial properties.

The hydrogel forming polymer aids in fiber formation and also acts as a gelling agent, allowing the mat to be placed and maintained at a surgical site, while also allowing for appropriate positioning. The PVOH may wet out during further processing or upon placement in an aqueous environment, such as internally within a mammal, becoming a more homogenous film. The PGS component affords anti-adhesive and antimicrobial characteristics. The fibrous production method makes possible the combination of PVOH and PGS in a workable form and helps with rapid hydration of the mat, aiding in its surgical placement.

Exemplary embodiments thus provide the advantage of a material that itself readily adheres to tissue, prevents adhesion between the tissue it separates, and has desirable wetting, handling and strength characteristics. Additionally, exemplary embodiments have hemo-compatibility for use in the presence of blood, maintain wet strength that permits them to be repositioned as necessary during placement, have antimicrobial properties that permit them to be used in locations having a presence or risk of infection, and have a sufficiently low degree of cross-linking such that they can still resorb in a relatively short time frame as desired.

Various features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 shows an image of an electrospun film in accordance with an exemplary embodiment.

FIG. 2 shows a portion of the image of FIG. 1 at greater magnification.

### DETAILED DESCRIPTION OF EXEMPLARY EMBODIMENTS

Provided herein are articles and processes of forming articles that can be used to reduce or prevent adhesion between adjacent tissues following surgical and other medical procedures that combine PGS and a hydrogel forming polymer, generally as a non-woven textile in the form of a fibrous mat or film. While the hydrogel forming polymer is discussed primarily herein with respect to PVOH, it will be appreciated that other biologically acceptable materials may be used in combination with or in place of PVOH, including, for example, hyaluronic acid, carboxymethylcellulose, hydroxymethyl cellulose, alginate, collagen, gelatin, and combinations thereof.

Pure PGS films can be difficult to use within the surgical field because PGS alone exhibits little to no adherence to tissue. The benefits of PGS still encourage its desirability for use and thus, such films can be sutured in place. However, sutures can themselves cause adhesions, so the use of sutures with adhesion barriers is generally sought to be avoided. Furthermore, when in a thin film form, PGS may not have sufficient strength to maintain suture fixation. A pure PGS thermoset film may also need extensive cross-linking to create a film that can be handled during the procedure. As a result of the extensive cross-linking, the film has a prolonged degradation profile, whereas the degradation window for an adhesion barrier is preferably between 2 to 4 weeks.

Exemplary embodiments include a fibrous mat that is a combination of PGS and PVOH useful in adhesion barrier and other applications and can provide a more suitable degradation window than pure PGS thermoset films, such as between 1 to 4 weeks, such as 2 to 4 weeks. The fibers of the mat are preferably formed by electrospinning and generally may be characterized as nanofibers, although it will be appreciated that cross-sectional diameters may vary as a result of the manufacturing process and further that cross-sectional diameters on the order of microns may intentionally be formed by changing processing conditions if desired.

The PGS is present for its resorptive and antimicrobial properties, as well as its effectiveness as a barrier. The hydrogel forming polymer, such as PVOH, is used as a temporary adhesive to enhance the adhesion of the device to the surrounding tissue but does so without stimulating tissue adhesions due to the presence of PGS. The presence of PVOH also reduces the need for extensive post-process crosslinking to produce a strong tack free film.

The PGS/PVOH fibers, once formed, do not require thermal cross-linking to eliminate the tackiness of the PGS resin as in pure PGS thermoset films. With the present approach, a PGS-based device is produced that maintains rapid degradation properties, unlike pure PGS thermoset films that have high cross-linking for mechanical strength, but which in turn results in a lengthy degradation that is undesirable for adhesion barrier applications.

The fibrous structure of the formed mat in accordance with exemplary embodiments also increases the available surface area to allow for rapid hydration, while at the same time providing mechanical strength to the device. The PVOH enhances the mat's ability to adhere to the tissues on opposing sides of the mat that the barrier is being used to separate.

The production of a PGS/PVOH structure to provide an adhesion barrier that has sufficient mechanical strength upon initial formation without the need for high cross-linking was unexpected and surprising. Without being bound by any theory, it believed that one or more mechanisms resulting from dipole interaction may play a role in the surprising results. Furthermore, analyses suggest that some localized cross-linking may be occurring between the PGS and PVOH during the electrospinning process.

With respect to dipole interaction, the fiber—formed by an electrospinning method as discussed subsequently in more detail—may result in a sheath-core fiber in which the PVOH forms a sheath around a PGS core. The electrical field present in the electrospinning process may act to align the two polymers so that their polar functional groups are hydrogen bonded, decreasing the potential for them to interact. PGS resin by itself is very sticky but when combined with PVOH and electrospun it loses the stickiness, which may be due to hydration of the PVOH. This allows for easy manipulation of the mat.

The electrospun fiber in a sheath-core suggests that the electric field is acting on the structural conformation of the polymers, causing them to phase separate.

The electrical field present in the electrospinning process may also or alternatively align the functional groups in the polymer; this reduces the activation barrier and provides enough energy to induce cross-linking, either through electrical or heat energy.

It was observed that a simple mixture of PGS-PVOH cast and dried produces different results from structures produced by electro-processing as described herein, as reflected in attenuated total reflectance Fourier-transform infrared (ATR FTIR) studies that shows a dominate presence of the PVOH OH-stretch in the FTIR spectrum which suggests that the fibers produced as described herein have undergone crosslinking between the PVOH and PGS during the electrospinning process.

Exemplary embodiments may be formed by first dissolving a blend of PGS and the hydrogel forming material (e.g. PVOH). It will be appreciated that PGS also includes PGS-based co-polymers and other constituents, such as a PGS+PVOH copolymer and/or a PGS+PEG (polyethylene glycol) copolymer, for example, in the blend along with the hydrogel forming polymer. In some embodiments, the PGS may be a PGS-pharmaceutical compound copolymer, such as a PGS-salicylic acid copolymer.

Any suitable solvent that dissolves both constituents of the blend and has a high vapor point may be used. Exemplary solvents include hexafluoroisopropanol (HFIP), dimethyl sulfoxide (DMSO), dimethylformamide (DMF), tetrahydrofuran (THF), ethyl acetate, methanol, ethanol, isopropanol, propyl acetate, acetone, methyl ethyl ketone (MEK), water, and combinations thereof.

The blend ranges from a solids content from 10% to 90% PGS by weight, with the balance of the solid content being PVOH (or other hydrogel forming polymer). It will be appreciated, however, that in some cases minor amounts of non-polymeric additives may also be present. In some embodiments, the weight blend is about 20% to about 80% PGS, about 30% to about 60% PGS, about 35% to about 65% PGS, about 40% to about 60% PGS, about 45% to about 55% PGS, or about 50% PGS, as well as any range, subrange, or number therebetween of the foregoing. In some embodiments, the weight ratio is 55:45 PVOH:PGS.

The PGS may range in weight-average molecular weight from about 2,000 Daltons to about 50,000 Daltons, typically between 5,000 Daltons and 25,000 Daltons, such as 10,000 Daltons to 15,000 Daltons, and any range, subrange or number therebetween of the foregoing. The PVOH may range in weight-average molecular weight from about 10,000 Daltons up to about 100,000 Daltons, such as up to about 80,000, up to about 60,000, up to about 40,000, up to about 25,000, and any range, subrange or number therebetween of the foregoing.

In order to process the solution, the total solids content (i.e., PGS+PVOH) of the solution ranges from about 2% to about 10% by weight, such as about 3%, about 4%, about 5%, about 6%, about 7%, about 8%, about 9%, or any range, subrange or number therebetween.

The polymers are preferably dissolved in the solvent by mechanical agitation and/or sonication. Once dissolved, the solution is ready for processing. The processing is preferably accomplished by electrospinning, although other methods, such as melt electrospinning (i.e. using a polymer blend directly in the absence of a solvent) or 3-D printing may also be employed. In some embodiments, an in-line twin screw or other type of extruder may be used to form a pre-polymerized sheath-core rod of a PVOH sheath and PGS core that can be used as feed stock for melt electrospinning and/or 3-D printing formation of a fibrous mat structure.

For electrospinning, the solution is loaded into a syringe or other reservoir with a needle attached; the needle gauge size should be between 12 and 25. The loaded reservoir is then coupled with a pump, such as placing into a syringe pump. A power source is attached that can supply a positive voltage to the needle attached to the reservoir and a negative voltage to a conductive collection device. The voltage difference can range from 5 kV to 70 kV, such as about 10 to about 50 kV, such as about 15 kV, about 20 kV, about 25 kV, about 30 kV, about 35 kV, about 40 kV, about 45 kV, and any range, subrange or number between any of the foregoing. In some embodiments, the power source is at a voltage between about 20 kV and 30 kV.

The collection device used in the electrospinning process may be either a stationary plate or a moving/rotating assembly. When both the needle and the collection device are attached to the power source and the needle is facing the collection device and the tip of the needle is at a distance of 5-20 cm from the collection device, the syringe pump is controlled to pump between a rate of 1 L/min and 200 μL/min. Once the pump is on and flowing, the power source can be turned on for the electrospinning process to occur.

In some embodiments, multiple syringes of material can be used concurrently to create thicker mats. Alternatively, or in combination, reservoirs can be replaced as their content is exhausted such that new layers are electrospun on top of the initial layers to create thicker films where desired. Typically, the final thickness of the mat will range from 30 μm to 500 μm; in one embodiment, the fibrous mat comprises PGS/PVOH blended nanofibers with a mat thickness of about 100 to about 200 μm. In some embodiments, the mat may be calendered to a desired thickness and/or to help provide a more homogenous film structure prior to implantation.

In some embodiments, multiple syringes may contain different materials that can be electrospun concurrently to form a mixed fiber mat. For example, individual streams of cells, and extra-cellular matrix (ECM) components; including collagen, laminins, fibronectin, vitronectin, elastin, proteins (including growth factors and hormones), glycosaminoglycans, proteoglycans and hyaluronan, chondroitin sulfate, dermatan sulfate, heparan sulfate, heparin, keratin sulfate, and matrix metalloproteinases, etc., could be co-spun with the PGS/PVOH blends to form a faux ECM structure. Furthermore, different syringes can be used at various times in the spinning process to produce layered mat structures.

When combining multiple streams of materials, the fiber structure for each of the materials may be the same or different, depending on the application. In some embodiments, loaded fibers (i.e. those containing an active or other additive) and unloaded fibers may need different nano structures. For example, unloaded fibers can be used to provide structure, while loaded fibers may have varying diameters to control the release rate of the actives or to protect biologics/cells for varying times. In some cases, for example, it may be desirable for a cell loaded fiber to have a larger average cross-sectional diameter than the unloaded fibers to protect the cells during the initial inflammatory response from an implant procedure, which would require a certain thickness that would retard degradation so that the cells were not released until after the inflammatory response decreases.

After the mat has been deposited, it may be peeled from the collection device. Conductive substrates and/or tapes which can bend may be adhered to or used as the collection device to facilitate removal of the electrospun mat. Alternatively, a device/substrate may be placed between the needle and the negative source that has surface properties that allow for easy removal of the electrospun mat; this device/substrate essentially “catches” the electrospun fiber as it is being attracted to the negative source.

In some embodiments, the mat is permanently deposited onto a substrate which is part of the final construct. The mat may be deposited onto a textile substrate which can impart anti-adhesion properties to the textile. The textile may be a knit, weave or braid in a flat or tubular form. In addition to the reduction of adhesions, the mat may act as a means to control the permeability of the textile structure. In some embodiments, the textile substrate is gauze on which the mat is applied and/or hydrated, annealed and dehydrated to the gauze for wound care.

The electrospun mat may thereafter optionally be thermoset to promote strength and longer material stability. It may be thermoset from 120-140° C. and from 0-48 hours, typically under an inert atmosphere.

In other embodiments, curing is accomplished by exposure of the formed mat/film to microwave radiation; other methods of curing include infrared (IR) blackbody curing and corona discharge (such as a peroxide driven crosslink as a result of the corona producing ultraviolet (UV) and ozone that could attach the PVOH), lyophilization, and gamma radiation.

Once formed and optionally cured, the electrospun film can then be used as an adhesion barrier in an open procedure in which a medical professional can manipulate the film with forceps and drape it over the area of interest, similar to a piece of cloth. Alternatively, the device can be loaded into a laparoscope or catheter and deposited/manipulated with the laparoscope.

It will be appreciated that a number of factors may be varied to achieve the desired characteristics of the finished mat, including fiber size, fiber density, fiber morphology, fiber composition, film thickness, cure time, molecular weights of polymer constituents, relative weight percentage of polymer constituents, and voltage drop, among others.

Articles formed in accordance with exemplary embodiments may also be used in other applications, such as wound care and drug delivery. For example, as the electrospinning process produces a stable film without the need for a high temperature cure, a variety of heat sensitive additives, such as actives, therapeutics, biologics, etc., could be incorporated into the fiber structure. Depending on where the drug partitions into the sheath-core structure would determine its release kinetics.

One example includes using the material to deliver a chemotherapeutic for high grade glioma treatment creating a preformed disc or moldable putty that could be easily placed at the treatment site without the need for prior gelation and/or to line or fill the cavity following tumor removal.

Fiber architecture and drug loading techniques can be manipulated in accordance with the articles of exemplary embodiments to achieve different drug release behaviors and/or polymer degradation behaviors.

In still other embodiments, the mats may be created in sizes that are large enough to resemble cloth that can be used to create other structural components, such as a pouch for use with pacemakers that could reduce infection and adhesions upon implantation into the tissue. The cloth concept can also be used in other textile composite constructions, as well as chronic diabetic wound dressings to provide both lubricity and reduced fibrosis. Applications for drug delivery and antimicrobial application for dermo-cosmetic and chronic skin conditions like psoriasis may also be realized with exemplary embodiments.

According to other embodiments, the mat can be formed into a film for use as a barrier laminate for single-use disposable containment to prevent wall sticking of cells or delivery of actives and nutrients. Because of wet-adhesion to tissue, composites of PGS-PVOH can also be used as a buccal or sublingual drug delivery device, including as an oral delivery device for active pharmaceutical ingredient (API) and cannabinoid actives, and/or for external application such as transdermal superficial drug delivery or other burns and wound care treatments.

Still another application for exemplary embodiments includes prosthetic devices, such as hydrating the mat followed by conformal vacuum contact to the prosthetic device followed by dehydration. Here the film can be manipulated to conformally cover the device.

As noted previously, without wishing to be bound by theory, the PGS and PVOH may exhibit some minor level of crosslinking in the electrospun needle head through the sebacic acid and PVOH groups that contribute to the surprisingly higher increase in film integrity compared to pure PGS films. This may occur by the OH of the PVOH crosslinking with the COOH groups of the sebacic acid group from PGS because the electrical energy at the point of discharge is great enough that it could force the crosslink. An ester carbonyl from the condensation of sebacic acid and PVOH may be formed, with some hydrogens expected to react from the PVOH to create ketone carbonyls; aldehyde carbonyls if the PVOH backbone breaks; and peroxides (—O—O—) off the OH on the PVOH.

This further means no catalyst or other cross-linking agent is required and that crosslinking is achieved by electrical energy transitioned to thermal energy while the solution is being affected by the charge in the needle. Electro-charging a melt-flow spinneret head or any other method that can create similar localized areas of concentrated electrical energy to drive interactions such as conformational arrangements and/or new bonds between resin constituents may also be used.

Even small amounts of crosslinking are beneficial; if used in heart valves or other co-blended films, even a slight crosslink as a result of the electrical storm at the needle can stabilize the composition such that any subsequent thermal residency keeps the polymer construct stable.

Varying the voltage may vary the electrical-to-thermal energy to drive the crosslink. Achieving a minor amount of cross-linking without the presence of a cross-linking agent has the additional advantage of reducing the risk of cytotoxicity and/or adverse immune response.

Heat generation at the needle tip as a function of input energy is expected to show the temperature is significantly higher as input energy increases. Conductivity of the solvent can be modified with organo-metallics, such as vitamin B12 or other biomolecules.

Although the total energy at the needle tip is up to around 30 kV or higher in some embodiments, the current is around 1 mA, so the electrical energy applied to the needle is around 30 Watts. While this is low on a macrolevel, a liquid solution with PGS and PVOH takes this energy and starts it moving so that most of the electrical energy is converted into kinetic energy of solution particles. The polymer particles (or smaller size, molecules) take in very large amounts of motion energy (in a molecular or nanolevel) so that these molecules heat up significantly, even if the needle itself does not. This further suggests something chemical is happening while polymer solution travels from the needle to the collector, or when in the needle. As a result, only a very small amount of mass is being converted to heat energy by the kinetic energy. Because the mass at or leaving the needle is very low, the temperature change is in turn very high. Furthermore, the orientation of the polymer constituents by the electric field forces the reactive functional groups into close proximity reducing the required activation energy required for reactivity.

The invention has been reduced to practice and is further described in the context of the following examples which are presented by way of illustration, not of limitation.

### EXAMPLE

A 55/45 w/w blend of PVOH:PGS was added to HFIP solvent at a total solids weight percent of 4%. The PGS weight-average molecular weight was about ˜15,000 Daltons and PVOH molecular weight ranged between 13,000 and 23,000 Daltons. The mixture was sonicated at >50° C. and periodically agitated until the polymer constituents were completely dissolved, which occurred in less than 2 hours.

The resulting solution was loaded into a syringe of an electrospin apparatus and a 19-gauge needle was attached to the syringe. Electrodes from the apparatus power source were attached to the needle and to a stationary conductive platen; the needle was positioned to face the conductive platen with the tip set 14 cm from the platen.

The solution was pumped from the syringe at a rate of 29 μL/min and the power source was turned on to a voltage of +/−23 kV.

The solution was then deposited on the platen. A variety of mat/film thicknesses were created, some of which required refilling/replacing the syringes with additional solution.

Once the electrospun mat was deposited to the desired thickness, the electrospun mat was peeled from the conductive platen and thermoset under a nitrogen atmosphere for 12 hours at 130° C.

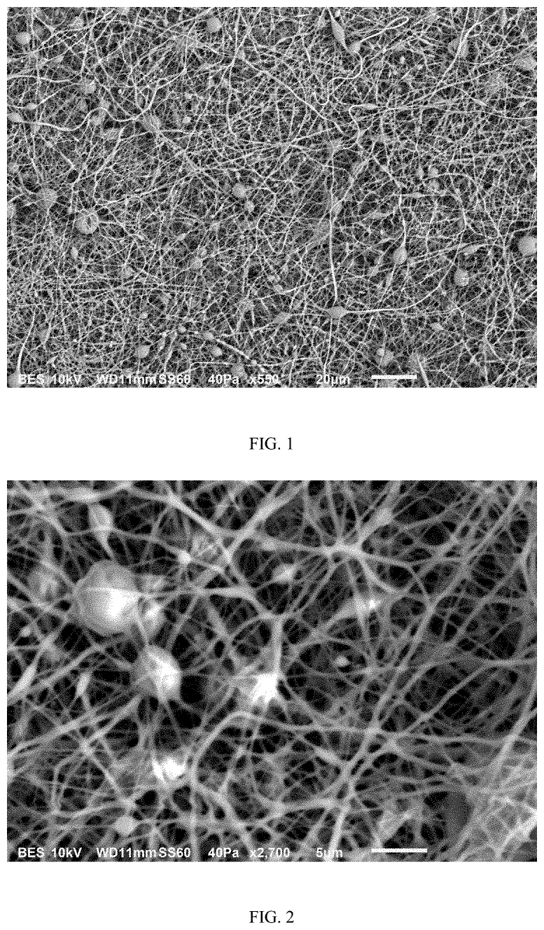

SEM images of the mats are shown in FIGS. 1 and 2.

The electrospun mats were subsequently used in a pre-clinical animal model to determine their efficacy in preventing abdominal adhesions. Female New Zealand white rabbits were used for this study. Briefly, after a midline laparotomy, an approximately 3×4 cm patch of parietal peritoneum and transversus abdominis muscle was removed from the right sidewall and circumscribed with a running suture of 2-0 silk. About a 10 cm length of the cecum was abraded 40 times with gauze. The electrospun mat was moistened slightly with saline and required no suture. The cecum was approximated to the sidewall and was approximated to the sidewall with two sutures (5-0 Prolene) placed through the inter-haustra serosal spaces of the cecum and placed on the lateral margin of the defect. The approximation was completed by the placement of two 5-0 Prolene sutures over the medial edge of the defect. In the control group, the defect was created, and the cecum and sidewall were approximated in the same fashion sans device.

The surgical site was evaluated at 13-15 days (“two weeks”) or 44-51 days (“seven weeks”) after surgery, and the extent and tenacity of adhesions to the defect were evaluated. The % of the defect area (in controls) or the area of either implant with adhesions was assessed, as was the % of the perimeter of the patch (or defect in controls) of either implant. The tenacity (as a 0-4 Grade score, where 0=no adhesions) of these adhesions was also assessed. Historical controls from a prior study were used for comparative purposes.

In all historical control animals, dense and tenacious adhesions formed between the cecum and 100±0% of the sidewall defect area at two and at six weeks. Using the electrospun mat, adhesions were reduced to 8±8% at two weeks and 40±31% at seven weeks. These differences may have been attributed to the wide variations in mat thickness between and within samples. There was a corresponding downward shift in the distribution of the tenacity of adhesions compared with historical controls at both timepoints.

The electrospun mat handled very nicely and although did not hold a suture well, it could be applied directly to tissue and with some slight moistening had some “tack” which obviated the need for sutures. The overall mild histological reaction to this material reflected its two-component nature. The more abundant laminated component was associated with a prominent fibrous capsule with minimal inflammation and some mineralization at seven weeks. The smaller and less abundant component evoked a low-grade chronic inflammation with giant cells at both time points. Some degradation was noted. The electrospun mat performed well in its adhesion prevention properties and mild histological response, as well as in its handling properties.

While the foregoing specification illustrates and describes exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method comprising
electrospinning a combination of poly(glycerol sebacate) (PGS) resin and a hydrogel forming polymer resin to form nanofibers; and
depositing the nanofibers onto a surface to form a fibrous mat.

2. The method of claim 1, wherein the combination is free of a cross-linking agent.

3. The method of claim 2, wherein the hydrogel forming polymer resin is poly(vinyl alcohol) (PVOH) and the nanofibers exhibit cross-linking between the PGS and PVOH upon deposition onto the surface.

4. The method of claim 3, wherein the PGS and PVOH are a blend.

5. The method of claim 4, wherein the PGS and PVOH are blended in a common solvent to form a solution for the electrospinning.

6. The method of claim 5, wherein the total solids content of the solution is in the range of about 2% to about 10% by weight.

7. The method of claim 6, wherein the nanofibers are electrospun from the solution being pumped at a rate of about 1 microliter per minute to about 200 microliters per minute.

8. The method of claim 1 comprising electrospinning at a voltage differential in the range of 5 kV to 70 kV.

9. The method of claim 1, wherein the surface onto which the nanofibers are deposited is a textile.

10. The method of claim 1, further comprising forming a pouch from the fibrous mat.

11. The method of claim 1, further comprising electrospinning a second set of nanofibers from a composition different than the combination of PGS and the hydrogel forming polymer and co-depositing the second set of nanofibers with the PGS-hydrogel forming nanofibers to form the fibrous mat.

12. An article comprising a fibrous mat of nanofibers made according to the method of claim 1.

13. The article of claim 12, wherein the hydrogel forming polymer is selected from the group consisting of PVOH, hyaluronic acid, carboxymethylcellulose, hydroxymethyl cellulose, alginate, collagen, gelatin, and combinations thereof.

14. The article of claim 13, wherein the hydrogel forming polymer comprises PVOH.

15. The article of claim 12, wherein the PGS has a weight average molecular weight in the range of about 2,000 Daltons to about 50,000 Daltons.

16. The article of claim 12, wherein the hydrogel forming polymer comprises PVOH having a weight average molecular weight in the range of about 10,000 Daltons to about 100,000 Daltons.

17. The article of claim 12, wherein the PGS has a weight average molecular weight in the range of about 10,000 to about 15,000 Daltons and the hydrogel forming polymer is PVOH having a weight average molecular weight in the range of about 10,000 Daltons to about 25,000 Daltons.

18. The article of claim 17, wherein the nanofibers are 40% to 60% by weight PGS and 60% to 40% by weight PVOH.

19. The article of claim 12, wherein the fibrous mat has a thickness of about 30 microns to about 500 microns.

20. The article of claim 19, wherein the fibrous mat has a thickness of about 100 microns to about 200 microns.

21. The article of claim 12, wherein the fibrous mat further comprises cellular materials.

22. The article of claim 12, wherein the fibrous mat further comprises an active ingredient.

23. A method comprising placing the article of claim 12 on a tissue surface within a mammalian body.

24. The method of claim 23 comprising placing the article as a barrier intermediate two adjacent tissue surfaces within the mammalian body.

25. A method comprising
electrospinning a combination of PGS resin and a hydrogel forming polymer resin to form a first set of nanofibers;
electrospinning a second material to form a second set of nanofibers;
co-depositing the first set of nanofibers and the second set of nanofibers onto a surface to form a fibrous mat.

26. The method of claim 25, wherein the second set of nanofibers has a larger average cross-sectional diameter than the first set of nanofibers.

27. The method of claim 26, wherein electrospinning the second material comprises electrospinning a blend of PGS, PVOH and cells to form the second set of nanofibers.

28. The method of claim 27 further comprising electrospinning a third material comprising collagen to form a third set of nanofibers and co-depositing the third set of nanofibers with the first and second sets of nanofibers to form the fibrous mat.

29. The method of claim 28 comprising forming the fibrous mat as a synthetic extra cellular matrix (ECM) material.
